(19) United States
(12) Patent Application Publication
(10) Pub. No.: US 2025/0065332 A1
(43) Pub. Date: Feb. 27, 2025

(54) NOZZLE AND DISPENSER FOR TIME-RESOLVED ELECTRON MICROSCOPY STRUCTURAL STUDIES

(71) Applicants: Alexandra Ros, Phoenix, AZ (US); Peter Wiktor, Phoenix, AZ (US); Abhik Manna, Tempe, AZ (US); Petra Fromme, Mesa, AZ (US)

(72) Inventors: Alexandra Ros, Phoenix, AZ (US); Peter Wiktor, Phoenix, AZ (US); Abhik Manna, Tempe, AZ (US); Petra Fromme, Mesa, AZ (US)

(21) Appl. No.: 18/815,708

(22) Filed: Aug. 26, 2024

Related U.S. Application Data
(60) Provisional application No. 63/578,827, filed on Aug. 25, 2023.

(51) Int. Cl. B01L 3/00

(52) U.S. Cl. CPC B01L 3/502784; B01L 3/502715; B01L 2300/0864; B01L 2400/0439

(57) ABSTRACT

A device for time-resolved electron microscopy includes a nozzle and a piezoelectric droplet generator. The nozzle includes a chamber defining an inlet and an outlet, a first channel in fluid communication with the inlet of the chamber, and a second channel in fluid communication with the chamber. The piezoelectric droplet generator is coupled to the first channel and to a sample source. The piezo-electric droplet generator is configured to generate and provide droplets of the sample to the chamber. The second channel is configured to provide a substrate solution to the chamber, and the droplets of the sample and the substrate solution mix in the chamber and form a fluid stream when exiting the outlet for capture on an EM slide for study.

### CROSS-REFERENCE TO RELATED APPLICATIONS

This application is a non-provisional of and claims the benefit of U.S. Provisional Patent Application No. 63/578,827, filed on Aug. 25, 2023, the contents of which are incorporated herein by reference.

### STATEMENT REGARDING FEDERALLY SPONSORED RESEARCH OR DEVELOPMENT

This invention was made with government support under 2031343 awarded by the National Science Foundation. The government has certain rights in the invention.

### BACKGROUND

Conventional scanning electron microscopy (SEM) or transmission electron microscopy (TEM) can be used to investigate a number of important biological and chemical structures and dynamics, however it is often essential to analyze systems at more strict timescales. Time-resolved electron microscopy (EM) is used when researchers want to connect their spatial data with efficient temporal resolution.

It would be desirable to have a system that can rapidly mix substrates with a protein sample prior to dispensing on an EM grid to allow time-resolved EM studies to gain insights in protein structure and reaction mechanisms.

### SUMMARY

The present disclosure describes embodiments of a nozzle and dispenser design for rapidly mixing substrates with a protein sample prior to dispensing on an EM grid. This configuration allows for time resolved EM studies to gain insights in protein structure and reaction mechanisms.

The present disclosure provides a nozzle and a dispenser for time-resolved EM structural studies of proteins. Conventional microfluidic mixers mix the various components prior to entering and exiting a dispensing nozzle whereas the disclosed design combines and mixes a protein sample and various reactants and substrates in the nozzle just before dispensing to allow for shorter time periods for time-resolved EM experiments of protein structure and reaction mechanisms. In some embodiments, the nozzle is 3D printed. In some embodiments, the nozzle is combined with a piezoelectric droplet dispenser. In some embodiments, the nozzle comprises a larger diameter droplet generator channel and a smaller diameter substrate channel that feeds to a single exit to create the droplets for the EM experiments.

In one embodiment, the present disclosure provides a device for time-resolved electron microscopy. The device comprises a nozzle and a piezoelectric droplet generator. The nozzle includes a chamber defining an inlet and an outlet, a first channel in fluid communication with the inlet of the chamber, and a second channel in fluid communication with the chamber. The piezoelectric droplet generator is coupled to the first channel and to a sample source, the piezo-electric droplet generator configured to generate and provide droplets of the sample to the chamber. The second channel is configured to provide a substrate solution to the chamber, and the droplets of the sample and the substrate solution mix in the chamber and form a fluid stream when exiting the outlet.

In an aspect, the device further includes a housing, and the nozzle is formed within the housing.

In another aspect, the housing includes a first surface, and the outlet is formed in the first surface.

In yet another aspect, the first channel of the nozzle includes an opening having a diameter of about 20 μm to about 1.0 mm.

In a further aspect, the first channel of the nozzle includes an opening having a diameter of about 0.74 mm.

In another aspect, the first channel of the nozzle includes an opening having a diameter of about 0.93 mm.

In yet another aspect, the first surface is planar.

In an aspect, the second channel of the nozzle includes an opening having a diameter of about 0.3 mm to about 0.5 mm.

In another aspect, the second channel of the nozzle includes an opening having a diameter of 0.39 mm.

In yet another aspect, the fluid stream is captured on one or more EM slides for electron microscopy study.

In another embodiment, the present disclosure provides a nozzle comprising a housing, a chamber formed within the housing, the chamber defining an inlet and an outlet, a first channel formed within the housing, the first channel integrally formed with the chamber and in fluid communication with the inlet of the chamber, and a second channel formed within the housing, the second channel integrally formed with the chamber and in fluid communication with the chamber. The chamber is configured to receive a sample delivered through the first channel from a droplet generator. The chamber also is configured to receive a substrate delivered through the second channel. The sample and the substrate mix in the chamber and exit through the outlet as a fluid stream.

In an aspect, the chamber includes a proximal end and a distal end, and wherein the first channel is coupled to the chamber at the proximal end.

In another aspect, the second channel is coupled to the chamber at the distal end.

In an aspect, the droplet generator is a piezoelectric droplet generator configured to generate and provide droplets of the sample to the chamber.

In another aspect, the first channel includes an opening having a diameter of about 20 μm to about 1.0 mm.

In yet another aspect, the first channel includes an opening having a diameter of about 0.74 mm.

In a further aspect, the first channel includes an opening having a diameter of about 0.93 mm.

In another aspect, the second channel includes an opening having a diameter of about 0.3 mm to about 0.5 mm.

In yet another aspect, the second channel includes an opening having a diameter of 0.39 mm.

In an aspect, the fluid stream is captured on one or more EM slides for electron microscopy study.

Other aspects of the disclosure will become apparent by consideration of the detailed description and accompanying drawings.

### BRIEF DESCRIPTION OF THE DRAWINGS

FIG. 1 is a perspective view of a microfluidic device for time-resolved electron microscopy structural studies of proteins according to an embodiment of the present disclosure.

FIG. 2A is a cross-sectional view of a nozzle of the microfluidic device illustrated in FIG. 1.

FIG. 2B is a perspective view of a piezoelectric droplet generator of the microfluidic device illustrated in FIG. 1.

FIG. 3 is a perspective view of a nozzle of the microfluidic device illustrated in FIG. 1 and FIG. 2A.

FIG. 4A is an image of an example nozzle according to an embodiment of the present disclosure.

FIG. 4B is an image of an experiment with the microfluidic device of the present disclosure demonstrating operability with dispensed droplets exiting the nozzle illustrated in FIG. 4A.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Unless otherwise defined, all technical and scientific terms used herein have the same meaning as commonly understood by one of ordinary skill in the art. In case of conflict, the present document, including definitions, will control. Example methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an,” and “the” include plural references unless the context clearly dictates otherwise. The present disclosure also contemplates other embodiments, “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about to about 4” also discloses the range “from 2 to 4.” The term “about” may refer to plus or minus 10% of the indicated number. For example, “about 10%” may indicate a rage of 9% to 11%, and “about 1” may mean from 0.9-1.1. Other meanings of “about” may be apparent from the context, such as rounding off, s, for example “about 1” may also mean from 0.5 to 1.4.

For each recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.4, 6.5, 6.6, 6.7, 6.8, 6.9, and 7.0 are explicitly contemplated.

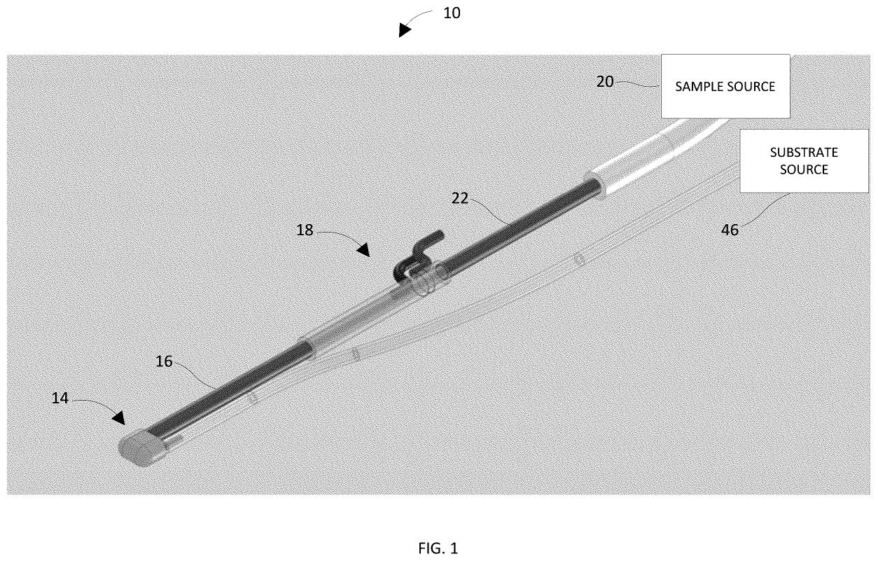

FIG. 1 illustrates a microfluidic device 10 for time-resolved electron microscopy structural studies of biomolecules (e.g., one or more proteins) in a sample. In one aspect, the microfluidic device 10 can be used for time-resolved cryo-electron microscopy. The microfluidic device 10 includes a nozzle 14 and a droplet generator 18. The droplet generator 18 is coupled to the nozzle 14 via a first conduit 16 and a sample source 20 via a second conduit 22. The droplet generator 18 is positioned upstream of the nozzle 14. The droplet generator 18 is configured to inject an aqueous solution (including the sample) in the form of droplets that are then directly dispensed from the nozzle 14 after mixing with a substrate solution. A substrate solution is a solution containing any chemical compound that might react with a particular biomolecule in the sample. For example, a protein is an enzyme, and the substrate would include the enzyme substrate dissolved in the solution.

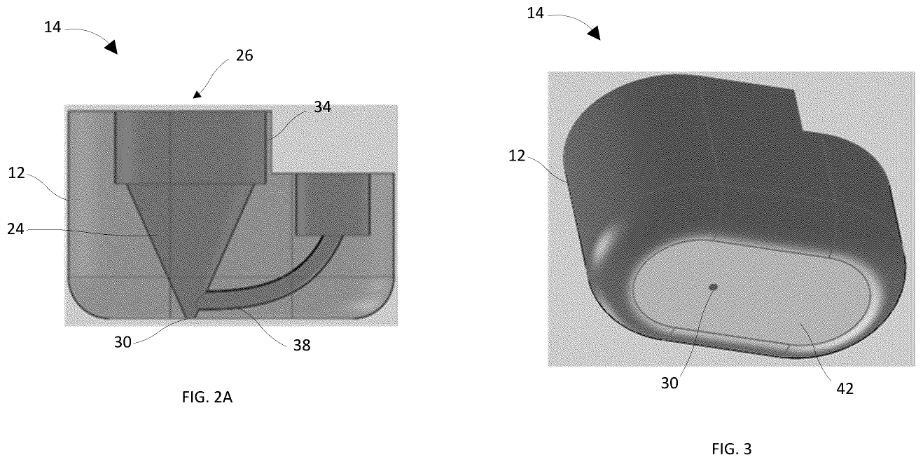

In one embodiment, the droplet generator 18 includes a piezoelectric droplet dispenser as illustrated in FIG. 2B. The piezoelectric droplet dispenser includes a piezoelectric actuator that expands or contracts at certain frequencies when a voltage is applied to the piezoelectric actuator to generate the droplets. For example, the piezoelectric actuator is excited a cosine wave signal with up to 87 volt amplitude and 12.5 kHz. Each droplet comprises an aqueous solution of the sample provided by the sample source.

With reference to FIG. 2A, the nozzle 14 is shown in more detail. The nozzle 14 is formed within a housing 12 and includes a chamber 24 having an inlet 26 and an outlet 30, a first channel 34 in fluid communication with the chamber 24, and a second channel 38 in fluid communication with the chamber 24. The second channel 38 is also in fluid communication with a substrate solution source 46. The chamber 24 is positioned at a distal end of the nozzle 14. The chamber 24 combines fluid from the first channel 34 and the second channel 38 just before or adjacent to the outlet 30. In other words, the chamber 24 combines or mixes the droplet containing the sample from the first channel 34 along with a substrate solution from the second channel 38. In one aspect, the nozzle 14 brings together a protein solution and a substrate solution, mixing quickly to be dispensed together (thereby initiating a reaction) by the nozzle 14. In time-resolved cryo-electron microscopy setting, the droplets dispensed from the nozzle 14 are frozen once deposited on an EM grid for sample analysis. This microfluidic device 10 would allow for time-resolved cryo-EM (as there is a certain delay between mixing/reaction initiation and depositing on the grid).

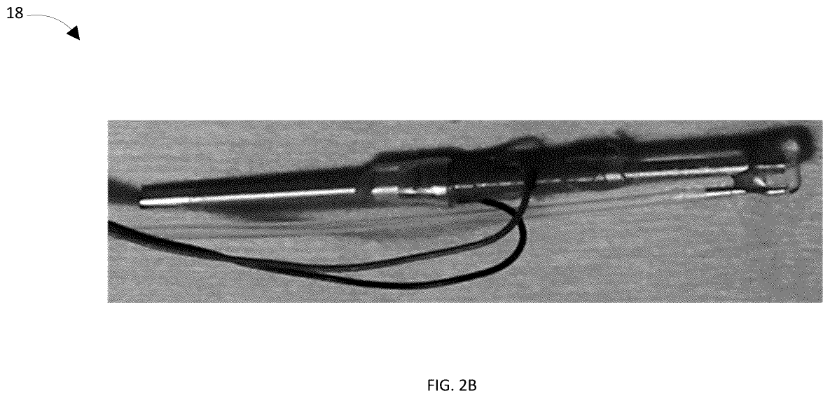

With reference to FIGS. 2A and 3, the housing 12 (defining the nozzle 14) is 3D printed in some embodiments. The first channel 34 is in fluid communication with the first conduit 16 to receive the droplets of the sample. The first channel 34 has an opening sized about 20 μm to about 1.0 mm inner diameter. In one aspect, the opening of the first channel is about 0.5 mm to about 1.0 mm inner diameter. In another aspect, the size of the opening of the first channel 34 is about 0.74 mm inner diameter. In another aspect, the size of the opening of the first channel 34 is about 0.93 mm inner diameter. The first channel 34 is integrally formed with the chamber 24. The chamber 24 is funnel-shaped where a diameter decreases from a proximal end of the chamber 24 to a distal end of the chamber 24. The outlet 30 is integrally-formed at a distal end of the chamber 24. The outlet 30 of the nozzle 14 is axially-aligned with the first channel 34 and is configured to provide a fluid stream of dispensed droplets containing a sample for EM studies to gain insights in protein structure and reaction mechanisms, for example. The housing 12 includes a surface 42 that is planar or flat in which the outlet 30 is formed. This surface provides improved droplet ejection and droplet formation.

With continued reference to FIGS. 2A and 3B, the second channel 38 feeds into the chamber 24. As shown, the second channel 38 provides a substrate solution into a distal portion of the chamber 24. The second channel 38 is positioned to allow for a rapid mixing of a protein solution and a substrate solution to thereby initiate a reaction just prior to exiting the outlet 30 of the nozzle 14. The second channel 38 has an opening sized about 20 μm inner diameter to about 0.5 mm inner diameter. In one aspect, the size of the opening of the second channel 38 is between about 0.3 mm and 0.5 mm inner diameter. In another aspect, the size of the opening of the second channel 38 is about 0.39 mm inner diameter.

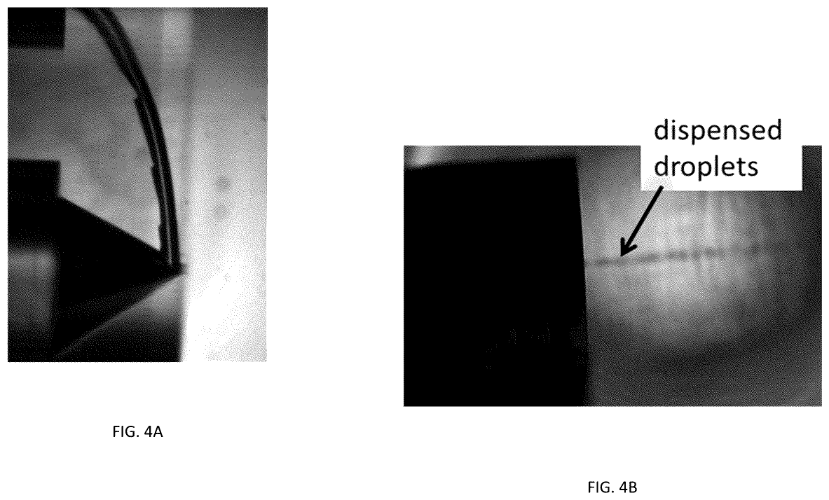

FIGS. 4A and 4B illustrate an example of a microfluidic device 10 according to an embodiment of the present disclosure. In particular, FIG. 4B illustrates a plurality of dispensed droplets exiting from the outlet 30 of the nozzle 14. These droplets are captured on EM slides for further study of the droplets.

Various features and advantages of the present disclosure are set forth in the following claims.

What is claimed is:

1. A device for time-resolved electron microscopy, the device comprising:
a nozzle including
a chamber defining an inlet and an outlet,
a first channel in fluid communication with the inlet of the chamber, and
a second channel in fluid communication with the chamber;
a piezoelectric droplet generator coupled to the first channel and to a sample source, the piezo-electric droplet generator configured to generate and provide droplets of the sample to the chamber;
wherein the second channel is configured to provide a substrate solution to the chamber, and
wherein the droplets of the sample and the substrate solution mix in the chamber and form a fluid stream when exiting the outlet.

2. The device of claim 1, further comprising a housing, and wherein the nozzle is formed within the housing.

3. The device of claim 2, wherein the housing includes a first surface, and wherein the outlet is formed in the first surface.

4. The device of claim 3, wherein the first channel of the nozzle includes an opening having a diameter of about 20 μm to about 1.0 mm.

5. The device of claim 4, wherein the first channel of the nozzle includes an opening having a diameter of about 0.74 mm.

6. The device of claim 4, wherein the first channel of the nozzle includes an opening having a diameter of about 0.93 mm.

7. The device of claim 3, wherein the first surface is planar.

8. The device of claim 1, wherein the second channel of the nozzle includes an opening having a diameter of about 0.3 mm to about 0.5 mm.

9. The device of claim 8, wherein the second channel of the nozzle includes an opening having a diameter of 0.39 mm.

10. The device of claim 1, wherein the fluid stream is captured on one or more EM slides for electron microscopy study.

11. A nozzle comprising:
a housing;
a chamber formed within the housing, the chamber defining an inlet and an outlet;
a first channel formed within the housing, the first channel integrally formed with the chamber and in fluid communication with the inlet of the chamber; and
a second channel formed within the housing, the second channel integrally formed with the chamber and in fluid communication with the chamber;
wherein the chamber is configured to receive a sample delivered through the first channel from a droplet generator,
wherein the chamber is configured to receive a substrate delivered through the second channel, and
wherein the sample and the substrate mix in the chamber and exit through the outlet as a fluid stream.

12. The nozzle of claim 11, wherein the chamber includes a proximal end and a distal end, and wherein the first channel is coupled to the chamber at the proximal end.

13. The nozzle of claim 12, wherein the second channel is coupled to the chamber at the distal end.

14. The nozzle of claim 11, wherein the droplet generator is a piezoelectric droplet generator configured to generate and provide droplets of the sample to the chamber.

15. The nozzle of claim 11, wherein the first channel includes an opening having a diameter of about 20 μm to about 1.0 mm.

16. The nozzle of claim 15, wherein the first channel includes an opening having a diameter of about 0.74 mm.

17. The nozzle of claim 15, wherein the first channel includes an opening having a diameter of about 0.93 mm.

18. The nozzle of claim 11, wherein the second channel includes an opening having a diameter of about 0.3 mm to about 0.5 mm.

19. The nozzle of claim 18, wherein the second channel includes an opening having a diameter of 0.39 mm.

20. The nozzle of claim 11, wherein the fluid stream is captured on one or more EM slides for electron microscopy study.
